(19) United States
(12) Patent Application Publication
(10) Pub. No.: US 2026/0368451 A1
(43) Pub. Date: Nov. 19, 2026

(54) NLP-POWERED LANGUAGE MODELING TECHNIQUES FOR THE CLINICAL DOMAIN

(71) Applicant: RAAPID, INC, Louisville, KY (US)

(72) Inventors: Amitava DAS, West Bengal (IN); Ningthoujam Dhanachandra SINGH, Manipur Dhana (IN); Raxitkumar GOSWAMI, Tragad, Ahmedabad (IN); Pinal PATEL, Mahesana (IN); Amit SHETH, Columbia, SC (US)

(73) Assignee: RAAPID, INC, Louisville, KY (US)

(21) Appl. No.: 19/208,594

(22) Filed: May 15, 2025

(51) Int. Cl. G06F 40/58; G06F 40/55; G16H 10/60

(52) U.S. Cl. CPC G06F 40/58; G06F 40/55; G16H 10/60

(57) ABSTRACT

A comprehensive overview of the objectives of the present invention are provided, which aims to enhance various aspects of clinical language processing. The primary focus is on developing stable and robust word embeddings that accurately represent semantics. To achieve this, the invention seeks to employ nuanced information extraction through relevant n-grams, improve syntactic understanding for complex dependencies, and achieve accurate sentence segmentation through a specialized tokenizer. Domain specific tokenization tailored for biomedical language and collaboration with clinical experts are also highlighted. The invention further aims to boost clinical natural language processing performance using advanced techniques, enable contextual comprehension of clinical language nuances, and strategically utilize BERT for optimized embeddings. Ultimately, the invention aims to redefine clinical natural language processing by revolutionizing segmentation and contextual understanding for enhanced clinical text analysis.

### FIELD OF THE INVENTION

The present invention relates to the domain of natural language processing (NLP) and focuses on advancing language modeling techniques for application in the clinical domain. Language modeling, a foundational aspect of NLP, involves the comprehension and generation of human language. More preciously, this inventive solution directly addresses significant challenges that currently undermine the efficacy and performance of language models within clinical applications.

### BACKGROUND OF THE INVENTION

The initial challenge that the present innovation addresses pertains to the inconsistency observed in neural word embedding techniques. While several recent articles have highlighted the potency of these tools in capturing the semantic and syntactic nuances within words, they have also unveiled a fundamental instability within these embeddings. This instability, particularly pronounced in language models tailored for clinical contexts, refers to the variability in word embeddings concerning their frequency or context within a specialized domain. Such inherent instability undermines the dependability of these embeddings, which is of utmost significance within the clinical sphere. Consequently, a focused approach is essential to ensure optimal performance and reliability.

The second challenge that this innovation confronts revolves around the imperative need for a heightened comprehension of syntactic dependencies. Syntactic dependency is intricately tied to unravelling the complex relationships between words within a sentence that dictate their grammatical and structural roles. These relationships encompass a myriad of aspects, from subject-verb connections and modifiers to object relationships. The accurate representation of syntactic dependencies is of paramount importance across diverse natural language processing (NLP) tasks, spanning machine translation and text summarization to sentiment analysis and question answering systems. However, achieving precise syntactic dependency capture remains intricate due to the multifaceted nature of human language and the diverse ways sentences can be structured.

In response, the present innovation strives to surmount these challenges by pioneering novel solutions that not only mitigate the instability of word embeddings within clinical contexts but also enrich the comprehension and integration of syntactic dependencies. These advancements hold the potential to significantly elevate the performance and reliability of language models within the clinical domain, thereby facilitating more accurate and effective applications of natural language processing for clinical purposes.

Further, large language models (LLMs) possess the capacity of handling or processing textual data related to healthcare thereby, addressing the challenges of the healthcare. Neural network-based models, which typically rely on embeddings or vectorization, often struggle to capture the subtle nuances crucial to the clinical context. This field is dense with named entities, proper nouns, and intricate relationships between drugs, compounds, and clinical factors, requiring models to have a deep understanding of these connections.

The information disclosed in this background of the disclosure section is only for enhancement of understanding of the general background of the invention and should not be taken as an acknowledgement or any form of suggestion that this information forms the prior art already known to a person skilled in the art.

### OBJECTIVE OF THE INVENTION

The principal objective of the present invention is to enhance the embeddings to develop stable and robust word embeddings for clinical language to accurately represent semantics.

Yet another objective of the present invention is to extract context-rich information from clinical narratives using relevant n-grams.

Yet another objective of the present invention is to improve syntactic understanding to enhance language models' recognition of complex syntactic dependencies in clinical text.

Yet another objective of the present invention is accurate sentence segmentation to create a specialized sentence tokenizer for precise segmentation of clinical notes.

Yet another objective of the present invention is using domain-specific tokenization to develop an in-house tokenizer tailored for biomedical language, accommodating unique challenges.

Yet another objective of the present invention is clinical expert collaboration to collaborate with clinical experts to define effective sentence segmentation criteria.

Yet another objective of the present invention is Superior NLP Performance to boost performance of clinical natural language processing tasks through advanced techniques.

Yet another objective of the present invention is contextual comprehension to facilitate language models to understand clinical language nuances for accurate interpretation.

Yet another objective of the present invention is Strategic BERT Utilization to leverage BERT for predictive and masked language modelling, optimizing embeddings.

Yet another objective of the present invention is to revolutionize clinical NLP to redefine clinical natural language processing with precise segmentation and rich contextual understanding.

These and other objectives and advantages of the present subject matter will be apparent to a person skilled in the art after consideration of the following detailed description taken into consideration with accompanying drawings in which preferred embodiments of the present subject matter are illustrated.

### SUMMARY OF THE INVENTION

The invention introduces a comprehensive innovation that seeks to amplify various dimensions of clinical language processing. At its core, the invention aims to establish an improved foundation for word embeddings in the clinical context, ensuring a strong and resilient representation of semantic meaning. To achieve this, the approach encompasses several elements, including nuanced extraction of information using relevant n-grams, enhanced grasp of syntactic intricacies to navigate complex language structures, and the creation of a dedicated sentence tokenizer to ensure precise sentence segmentation. Of significant note are the tailored tokenization techniques for biomedical language and collaboration with clinical experts, which constitute crucial aspects of the invention.

Additionally, the patent application emphasizes the aspiration to elevate the performance of clinical natural language processing through the application of advanced methodologies. This endeavour involves empowering language models to fathom the contextual nuances embedded within clinical language, thereby facilitating more accurate interpretation. The strategic integration of BERT, an advanced language model, plays a pivotal role in refining embeddings by employing techniques such as predictive and masked language modelling.

The core focus of the present invention is to substantially improve the realm of clinical language processing through a series of well-defined objectives. Firstly, it aims to elevate the quality of embeddings, specifically cultivating stable and resilient word embeddings that excel in representing the intricate nuances of clinical language with utmost precision. Another pivotal aim is the extraction of information imbued with contextual significance from clinical narratives, employing pertinent n-grams to uncover rich layers of meaning.

Enhancing the understanding of syntactic complexities within clinical text is another vital facet of the invention, bolstering the capabilities of language models to adeptly recognize intricate syntactic dependencies.

Moreover, a specialized sentence tokenizer is targeted for development, ensuring that the segmentation of clinical notes is executed with unparalleled accuracy. To address the distinctive challenges posed by biomedical language, a domain-specific tokenization approach is adopted, involving the creation of an in-house tokenizer tailored to accommodate the unique characteristics of this field. Collaboration with clinical experts is another distinguished goal, fostering effective criteria for sentence segmentation in partnership with those deeply versed in the clinical domain.

Furthermore, the invention aspires to achieve superior performance in the realm of clinical natural language processing through the strategic application of advanced techniques. This entails enabling language models to grasp the subtle nuances inherent in clinical language, thereby enabling a more accurate and insightful interpretation of the text. The astute utilization of BERT, a pioneering language model, takes centre stage in optimizing embeddings through predictive and masked language modelling methodologies.

Ultimately, a transformative goal is set forth: to revolutionize clinical natural language processing. This involves a comprehensive overhaul of segmentation strategies and the cultivation of a profound contextual understanding, reshaping the landscape of how clinical text is analysed and comprehended. In summation, the invention's objectives form a cohesive tapestry, woven with the threads of innovation and precision, with the overarching mission to redefine and elevate clinical natural language processing to unprecedented heights.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The patent or application file contains at least one drawing executed in color. Copies of this patent or patent application publication with color drawings will be provided by the Office upon request and payment of the necessary fee.

The drawings constitute a part of this invention and include exemplary embodiments of the present invention illustrating various objects and features thereof.

FIG. 1(a) illustrates a flow chart for NLP-powered language modeling techniques for the clinical domain;

FIG. 1(b) illustrates a working methodology of the NLP-powered language modeling techniques for the clinical domain;

FIG. 2 illustrates a knowledge graph while the central node is radiation therapy;

FIG. 3 illustrates graphical representation of a flip rotary positional encoding for extracting relative positions of the word in with in the sequence;

FIG. 4(a) illustrates words having more discrete numbers and thus more stability shown in red shades; and

FIG. 4(b) illustrates depicts unstable words represented more in blue shades.

### DETAILED DESCRIPTION OF THE INVENTION

The following description describes various features and functions of the disclosed apparatus. The illustrative aspects described herein are not meant to be limiting. It may be readily understood that certain aspects of the disclosed apparatus can be arranged and combined in a wide variety of different configurations, all of which are contemplated herein.

The following description of preferred embodiments of the invention is not intended to limit the invention to these preferred embodiments, but rather to enable any person skilled in the art to make and use this invention.

These and other features and advantages of the present invention may be incorporated into certain embodiments of the invention and will become more fully apparent from the following description as set forth hereinafter.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a,” “an,” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, integers, steps, components or groups thereof.

While the embodiments of the disclosure are subject to various modifications and alternative forms, specific embodiments thereof have been shown by way of example in the figures and will be described below. It should be understood, however, that it is not intended to limit the disclosure to the particular forms disclosed, but on the contrary, the disclosure is to cover all modifications, equivalents, and alternatives falling within the scope of the disclosure.

The present invention relates to a method which is adaptable to achieve nuanced information extraction from the textual data present in the clinical documents. The invention excels in extracting context-rich information from clinical narratives by harnessing relevant n-grams. The primary aim of the present invention is to enhance embeddings, yielding stable and robust word embeddings tailored for clinical language. These embeddings aptly capture semantics, ensuring an accurate representation of the text.

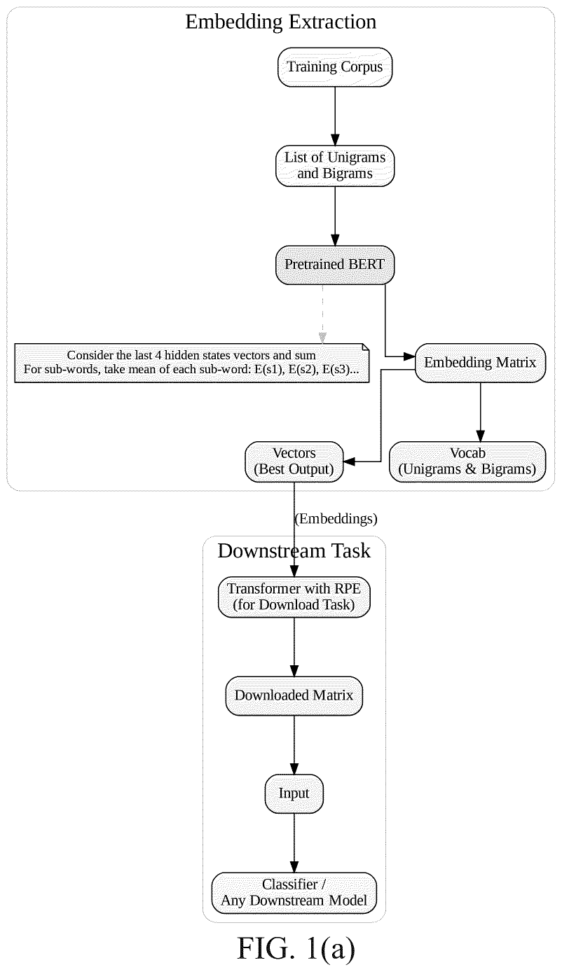

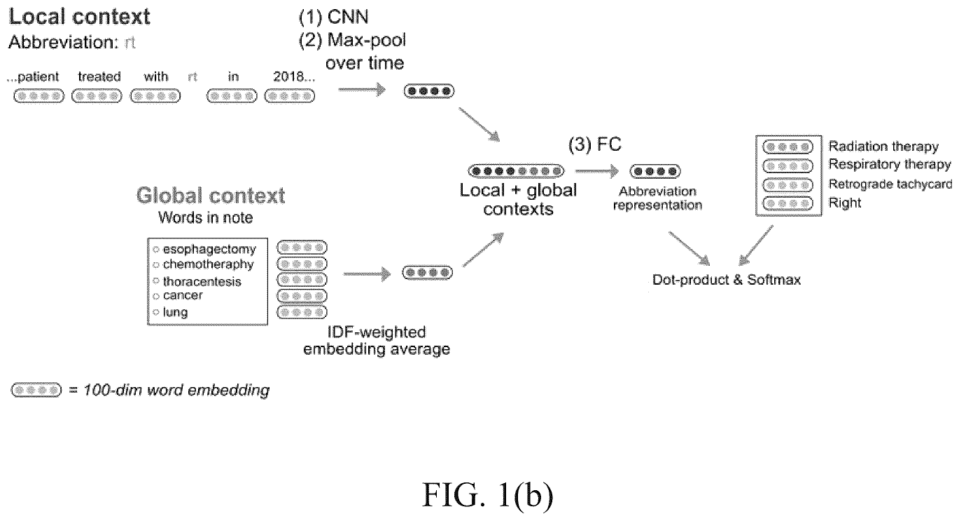

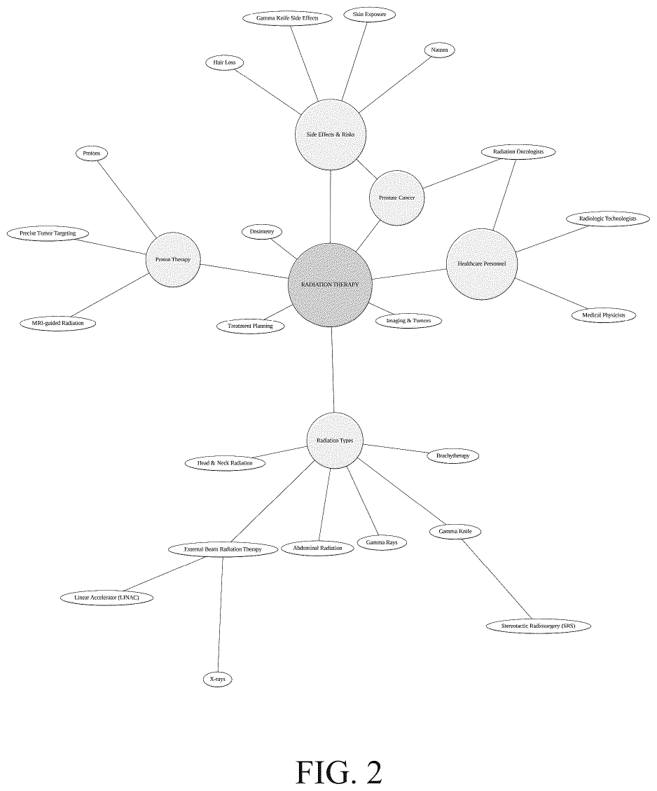

The present invention relates to a method formulated by integrating two distinct paradigms referring as neuro-symbolic approach. The neuro-symbolic approach integrate biomedical knowledge graphs with neural model which significantly improve performance, ensures stability and accurate representations of biomedical entities and relationships, as shown in FIGS. 1(a)-1(b) and FIG. 2.

In an exemplary embodiment, note such as “patient treated with rt in 2018” having a word RT that may have multiple interpretations within a healthcare context. To accurately determine the meaning in a given scenario, the proposed language modeling technque perform disambiguation at local and global levels. Local cues refer to the immediate context, such as surrounding words, while global cues encompass broader elements, like the overall topic of the document or conversation, as shown in FIG. 1. Different colors of the FIG. 1, distinguish local and global contexts, making it easier to follow their independent processing paths. The combined representation is visually highlighted, showing how these contexts merge into the final abbreviation representation. Colors also emphasize key operations, outputs, and the flow of information, reducing cognitive load and helping readers quickly understand complex relationships. The figure would be harder to interpret without color, leading to potential confusion.

FIG. 2 illustrates a network diagram for the word “rt” and the associated topics. Further, the central node represented by the yellow is the central or main concept and the node represented in green color are the associated topics of the central node. Further, color is crucial in this network diagram to differentiate the central concept (For example, in the present case central concept is “Radiation Therapy,” which is highlighted in yellow) from related nodes (highlighted in green), which represent associated topics. This color distinction helps emphasize the hierarchical relationship and central importance of the yellow node. Additionally, using the same color (such as green) for related nodes groups them visually, aiding in identifying clusters or thematic groupings. Without color, the central focus and connections would be less intuitive, making it harder to interpret the relationships and navigate the diagram efficiently.

The method technique offers a far more robust and precise solution than relying solely on traditional LLMs. Further, the method involves positional encoding which describes dependencies between tokens. Further, the method involves the flip-rotary positional encoding (RoPE).

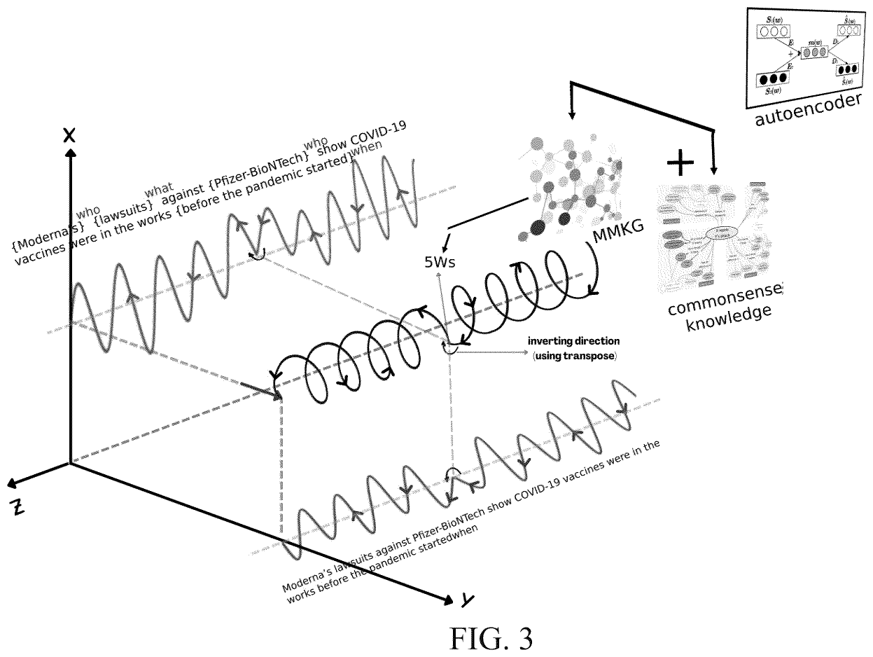

Further, positional values are generated by using rotation matrices on the embedding vectors. The rotation negates any absolute positional information and only retains information about the relative angles between every pair of word embeddings in a sequence. The dot product between two vectors is a function of the magnitude of individual vectors and the angle between them. Further, the intuition for the flip-rotary positional encoding (RoPE) is to represent the embeddings as complex numbers and the positions as pure rotations appied to them, as shown in FIG. 3.

Further, the integration of the graph embeddings into Large Language Models (LLMs) using Flip-Rotary Positional Encoding. As the LLM generates tokens sequentially and detects a designated parse token, triggering a flip in the rotational positional encoding. This flip enables the seamless fusion of SDNE-based graph embeddings with the LLM's vector space. Consequently, this integration enhances the stability and accuracy of the generated output, reducing errors during the generation process. The various colors shown in FIG. 3 differentiate the various stages and transformations in the 3D visualization. Each color (e.g., red, black, blue) represents a distinct part of the process, such as different contexts, pathways, or transformations (e.g., the inversion using transpose). Further, such distinct color representation allows viewers to follow how information flows and changes direction across dimensions. Without color, the overlapping trajectories and lines would blend together, making the relationships between components and their transformations unclear and significantly harder to interpret.

Mathematically, the formulations for a simple 2-dimensional case are defined as follows:

f_{Q}(xᵢ,i)=(W_{Q}xᵢ)e^{\sqrt{-1}iθ}

f_{Q}(xⱼ,j)=(W_{K}xⱼ)e^{\sqrt{-1}jθ}

g(xᵢ,xⱼ,i−j)=Re[(W_{Q}xᵢ)(W_{K}xᵢ)*e^{\sqrt{-1}(i-j)θ}]

where Re[ ] is the real part of a complex number and (w)*represents the conjugate complex number of (wₖ xᵢ). θ∈R is a preset non-zero constant. Formulating as a matrix (Q,K) multiplication, we get-

$${f}_{Q} ( {x}_{i} , i ) = ( \begin{matrix}cos m {θ}_{1} & - sin m {θ}_{1} \\ sin m {θ}_{1} & cos m {θ}_{1}\end{matrix} ) ( \begin{matrix}{W}_{Q , K}^{( 11 )} & {W}_{Q , K}^{( 12 )} \\ {W}_{Q , K}^{( 21 )} & {W}_{Q , K}^{( 22 )}\end{matrix} ) ( \begin{matrix}{x}_{i}^{( 1 )} \\ {x}_{i}^{( 2 )}\end{matrix} )$$

where

$$( {x}_{i}^{( 1 )} , {x}_{i}^{( 2 )} )$$

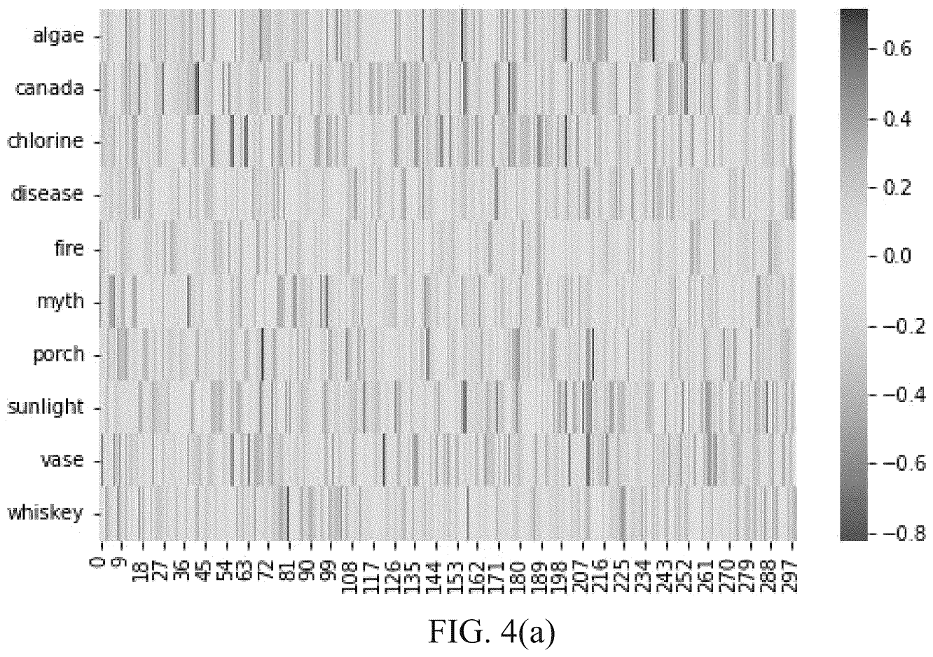

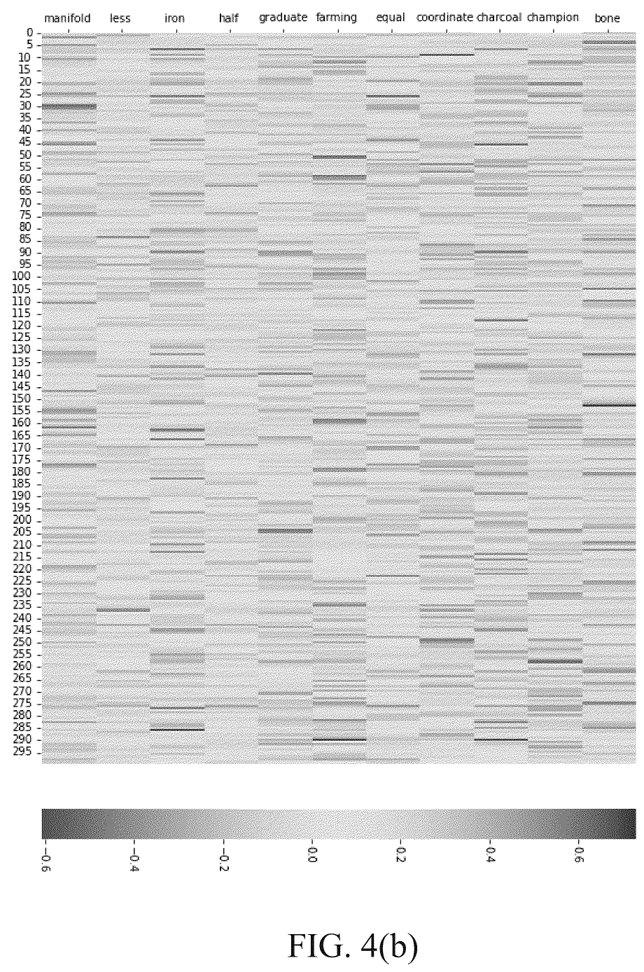

is xᵢ expressed in the form of 2D coordinates. In the same way, function g turn into matrix form. By rotating the transformed embedding vector by an angle in multiples of its position index, the model is adaptable to incorporate relative position information. Due to such characteristic, the method is termed as Rotary Position Embedding. In order to generalize the result in 2D to any xᵢ in where R_{d} is even, they divide the d-dimension space into d 2 sub-spaces and combine them in merit of the linearity of inner product, turning the attention formulation

$${f}_{Q , K} = {e}_{ij}^{rotary} = \frac{1}{\sqrt{d}} {( {RM}_{Θ , i}^{d} {W}^{Q , 1} ( {x}_{i} ) )}^{T} ( {RM}_{Θ , j}^{d} {W}^{K , 1} ( {x}_{j} ) )$$

$$RM = ( \begin{matrix}cos m {θ}_{1} & - sin m {θ}_{1} & 0 & 0 & … & 0 & 0 \\ sin m {θ}_{1} & cos m {θ}_{1} & 0 & 0 & … & 0 & 0 \\ 0 & 0 & cos m {θ}_{2} & - sin m {θ}_{2} & … & 0 & 0 \\ 0 & 0 & sin m {θ}_{2} & cos m {θ}_{2} & … & 0 & 0 \\ \cdot & \cdot & \cdot & \cdot & … & \cdot & \cdot \\ \cdot & \cdot & \cdot & \cdot & … & \cdot & \cdot \\ 0 & 0 & 0 & 0 & … & cos m {θ}_{d / 2} & - sin m {θ}_{d / 2} \\ 0 & 0 & 0 & 0 & … & sin m {θ}_{d / 2} & cos m {θ}_{d / 2}\end{matrix} )$$

where RM is orthogonal and sparse matrix predefined parameters Θ=θi=10000−2(i−1)/d, i∈[1, 2, . . . , d/2]. In contrast to the additive nature of the position embedding methods used by other works, their approach is multiplicative. Moreover, RoPE naturally incorporates relative position information through rotation matrix product instead of altering terms in the expanded formulation of additive position encoding when applied with self-attention, as shown in FIG. 4(a-b).

Color in this FIG. 4 represent the magnitude and direction of the values in the heatmaps. The gradient from blue to red encodes negative to positive values (or other opposing categories), enabling quick identification of patterns, trends, and contrasts across the data. Without color, the subtle variations in intensity would be lost, making significantly harder to interpret the differences or correlations within and between the heatmaps. The use of color also ensures that variations are easily perceptible, enhancing the figure's usability for analysis.

Further, the method involves syntactic and semantic understanding of the textual information. This involves enhancing language models' capability to recognize intricate syntactic dependencies within complex clinical text. Further, word embeddings are low-dimensional, dense vector representations that capture the semantic properties of words and serve as the foundation for modern neural language models. While word embeddings are widely applied in NLP, their stability provides an understanding of the text. Typically, a word embedding encodes the meaning of a word based on its frequency in the dataset and the context of surrounding words. As expected, words that occur less frequently tend to have lower stability, while more frequent words exhibit higher stability. However, words with medium-frequency show significant variance in stability.

Further, the stability, of the word, is defined as the percent overlap between nearest neighbors in an embedding space. Given a word W and two embedding spaces A and B, take the ten nearest neighbors of W in both A and B. Let the stability of W be the percent overlap between these two lists of nearest neighbors. 100% stability indicates perfect agreement between the two embedding spaces, while 0% stability indicates complete disagreement. In order to find the ten nearest neighbors of a word W in an embedding space A, we measure distance between words using cosine similarity. This definition of stability can be generalized to more than two embedding spaces by considering the average overlap between two sets of embedding spaces. Let X and Y be two sets of embedding spaces. Then, for every pair of embedding spaces (x, y), where x∈X and y∈Y, take the ten nearest neighbors of W in both x and y and calculate percent overlap. Let the stability be the average percent overlap over every pair of embedding spaces (x, y).

In an exemplary embodiment, Tabulated Chart 1, shows the top ten nearest neighbors for the word international in three randomly initialized word2vec embedding spaces trained on the NYT Arts domain. These models share some similar words, such as metropolitan and national, but there are also many differences. On average, each pair of models has four out of ten words in common, so the stability of international across these three models is 40%.

Tabulated chart 1: Top ten most similar words for the word international in three randomly initialized word2vec models trained on NYT arts domain:

| Model 1 | Model 2 | Model 3 |
|---|---|---|
| metropolitan | ballet | national |
| national | metropolitan | ballet |
| egyptian | bard | metropolitan |
| rhode | chicago | institute |
| society | national | glimmerglass |
| debut | state | egyptian |
| folk | exhibitions | intensive |
| reinstallation | society | jazz |
| chairwoman | whitney | state |
| philadelphia | rhode | exhibitions |

Instability is higher in Biomedical domain—Instability is more pronounced in the biomedical domain due to its abundance of named entities, proper nouns, and complex relationships between drugs, compounds, and clinical factors. These terms often occur infrequently, and their contextual usage varies significantly across cases. As a result, word embeddings in the biomedical domain tend to be highly unstable, making it more challenging to capture consistent and accurate semantic representations.

Precise sentence segmentation is yet another goal. The invention pioneers a specialized sentence tokenizer designed for accurate segmentation of clinical notes. In its pursuit of domain-specific tokenization, the invention creates an inhouse tokenizer customized for biomedical language, adeptly accommodating its unique challenges.

Collaboration with clinical experts constitutes another vital objective. The invention collaborates closely with clinical experts to establish effective sentence segmentation criteria.

The invention aspires for superior NLP performance. Through the implementation of advanced techniques, it enhances the performance of clinical natural language processing tasks.

Facilitating contextual comprehension is also a focal point. The invention empowers language models to discern the nuances of clinical language, thus enabling accurate interpretation.

Strategic BERT utilization is another key aim. By leveraging BERT for predictive and masked language modelling, the invention optimizes embeddings.

Ultimately, the invention strives to revolutionize clinical NLP. It redefines clinical natural language processing through precise segmentation and a profound grasp of contextual nuances, thereby paving the way for a new era in this field.
1. Training Corpus: The cornerstone of the invention innovative language model is a substantial training corpus comprising an extensive collection of million clinical notes accumulated over several years. This clean electronic corpus has been meticulously assembled through legal acquisition from reputable data companies, securing a comprehensive and authoritative clinical dataset. Originating from diverse healthcare institutions across the United States, these records provide a robust foundation for the invention language model's development.

To ensure the utmost comprehensiveness and representativeness of the invention training data, a multifaceted approach was adopted. Various clinical specialties are thoughtfully considered in order to encompass a wide array of medical domains and practices. This comprehensive coverage was extended to encompass the inclusion of different types of clinical documents, catering to the varied nature of medical records. Moreover, a careful analysis of physician and hospital distribution has been undertaken to capture distinct documentation patterns reflective of the real-world clinical landscape.

In addition to addressing the complexities of clinical specialties and document types, we acknowledged the significance of diverse patient demographics. Factors such as geographic location, age, gender, and patient type were meticulously accounted for to avoid potential bias in the training dataset. This collective consideration ensures that the invention language model is equipped with the broadest possible foundation of clinical knowledge, fostering accurate and unbiased representations within the clinical domain.
2. List of n-grams: From this extensive training corpus, we engaged in a meticulous extraction process to identify and isolate contextually relevant n-grams specific to the clinical domain. Employing the Unified Medical Language System (UMLS) as a guiding framework, we systematically extracted pertinent n-grams that encapsulate the intricate relationships and terminologies inherent to clinical narratives.

The significance of n-grams, particularly bigrams, becomes evident in their ability to convey nuanced meanings that transcend the sum of their individual components. For instance, the amalgamation of terms like “bowel sound” carries a deeper understanding of bodily function and related medical contexts that would be missed when considering individual unigrams. This strategic reliance on n-grams recognizes their capacity to encode richer contextual information, enabling enhanced comprehension of complex clinical narratives. In contrast, conventional Named Entity Recognition (NER) methods predominantly focused on unigrams tend to overlook the holistic insight offered by n-grams, making the invention approach substantially more informative in the clinical context.
3. Leveraging Pretrained BERT: Central to the invention innovation is the utilization of the Bidirectional Encoder Representations from Transformers (BERT) model, a state-of-the-art language representation technique. Through the incorporation of BERT's capabilities in next-sentence prediction and masked language modelling (MLM), we enhance the invention language model's ability to grasp the intricacies of clinical language.

The deployment of the uncased variant of the BERT model facilitates a cohesive merging of unigrams and relevant n-grams with BERT embeddings. This amalgamation unifies the outputs from both next-sentence prediction and MLM, forging a novel architecture tailored precisely for the clinical domain. This unique approach reinforces the invention language model's capacity to comprehend the nuances of clinical text, thereby underpinning the heightened efficacy of the invention innovation.

Dataset creation from a pretrained Transformer Language Model having a pivotal aspect of the invention methodology is the creation of datasets that encapsulate the outcomes of next-sentence prediction and the derived embeddings from the language model. The adoption of the BERT base uncased model presents a streamlined embedding approach, treating token and bigram case variations as equivalent. This unified case-insensitive treatment aids in contextual comprehension and compact embedding representation, thus enhancing the efficiency of the invention model while preserving the essence of clinical language.
4. Dataset Creation for Model Training: Tokenization is performed for segmenting clinical notes into coherent units for further processing. Unlike conventional punctuation-based methods, which fall short in the clinical context, the technique redefine sentence segmentation criteria. Specifically, the technique identify a double line space (equivalent to two line breaks) as the criterion for sentence demarcation. The NLP-powered language modeling techniques addresses the irregular sentence structure often found in clinical notes, circumventing challenges posed by non-standardized punctuation cues.

### According to Embodiment and Challenges:

### Tokenization Challenge for Acronyms:

In clinical notes, the utilization of acronyms such as “C.K.B” presents a tokenization challenge. Commonly used tokenizers like SpaCy or NLTK tend to break it into separate tokens at the full stop, treating “C” as one token, “K” as the next, and “B” as the last. In contrast, our in-house tokenizer recognizes the significance of “C.K.B” as a single entity, interpreting it as “creatine kinase B-type.” This tokenization challenge underscores the need for context-aware tokenization methods.

### Context-Based Casing and Abbreviation Understanding:

In the clinical domain, text interpretation may vary based on context, while in other instances, it remains consistent regardless of casing. Accurately discerning these nuances is paramount for the precise analysis and interpretation of clinical data.

For example, consider the medication “folfox.” In this context, whether it is written as “folfox” or “FOLFOX,” both forms refer to the same medication, and the meaning remains constant. This consistency facilitates reliable comprehension of the intended medication in clinical documentation.

Conversely, let's examine “AKA” versus “aka.” In the clinical domain, “AKA” specifically signifies “above-knee amputation,” a medical procedure. In general usage, “aka” stands for “also known as,” a non-clinical entity. This distinction underscores the necessity of considering context and domain when deciphering abbreviations or acronyms. The same term can have entirely different meanings based on casing, emphasizing the need for context-specific analysis within the clinical domain.

Similarly, consider “CA” versus “Ca.” In a clinical document stating, “The patient has CA,” “CA” refers to “cancer,” a medical condition. However, if the sentence were written as “The patient's Ca level dropped,” the change in casing of the second letter transforms “Ca” into “calcium.” This differentiation illustrates how the interpretation of the same abbreviation can shift based on context and casing.

These examples underscore the critical importance of context-aware analysis in the clinical domain. While some terms maintain consistent meanings regardless of casing, others exhibit varying interpretations depending on the context. Understanding these variations is essential for precise comprehension and interpretation of clinical text, empowering researchers, practitioners, and systems to extract meaningful insights and make informed decisions in healthcare settings.

### Version:

The innovation at hand takes on critical challenges within the intricate landscape of natural language processing (NLP), with a specific focus on the demanding realm of clinical contexts. These challenges encompass two fundamental aspects: the instability observed in neural word embedding techniques and the imperative need for an enriched understanding of syntactic dependencies.

### Addressing Embedding Instability:

The initial challenge tackled by this innovation is rooted in the instability exhibited by neural word embedding techniques. While these techniques have proven exceptional in capturing the nuanced semantics and syntactic structures of words, recent scrutiny has unveiled a potential drawback. Particularly pronounced in language models engineered for clinical domains, this instability stems from the fluctuations in word embeddings based on their frequency or contextual occurrences within specialized fields. This variability threatens the consistency and reliability of these embeddings, a concern that holds profound significance in the intricate world of clinical language. In response, the innovation is meticulously calibrated to stabilize and optimize word embeddings, ensuring their suitability for portraying precise and accurate semantics within clinical narratives.

### Enhancing Syntactic Dependency Comprehension:

The second pivotal challenge that this innovation grapples with revolves around the pressing necessity for a heightened comprehension of syntactic dependencies. Syntactic dependencies entail the intricate web of relationships governing the roles of words within a sentence, dictating their grammatical and structural significance. This comprehension holds pivotal importance across a spectrum of NLP applications, spanning from machine translation and text summarization to sentiment analysis and question-answering systems. However, the intricate nature of human language and the diverse ways in which sentences can be structured render the task of capturing these dependencies with precision a formidable undertaking. To surmount this challenge, the innovation endeavors to enrich the understanding and seamless integration of syntactic dependencies, thereby paving the way for more accurate and reliable language models specifically tailored for clinical NLP. In essence, the overarching aspiration of this innovation is to redefine and elevate the capabilities of language models within clinical contexts. By adeptly addressing the instability of word embeddings and amplifying the comprehension of syntactic dependencies, the innovation strives to bolster the accuracy and efficacy of natural language processing applications within clinical settings. This, in turn, holds the potential to revolutionize critical domains such as medical research, diagnosis, and healthcare practices, as language models gain the ability to more precisely interpret and analyze intricate clinical narratives.

### Tackling Embedding Instability:

The first challenge this innovation squarely confronts is the instability that sometimes plagues neural word embedding techniques. While these techniques exhibit impressive capabilities in capturing the semantic and syntactic intricacies within words, recent investigations have revealed an underlying concern. This instability, particularly pronounced when applied to language models crafted for clinical domains, arises due to the variance in word embeddings influenced by factors like frequency and contextual prevalence within specific domains. Such fluctuations undermine the consistency and reliability of these embeddings, a matter of immense significance in the intricate realm of clinical language. The innovation steps forward to counter this challenge through a meticulous calibration process, aiming to stabilize and optimize word embeddings. The objective is to ensure that these embeddings can reliably portray precise and accurate semantics within the context of clinical narratives.

### Advancing Syntactic Dependency Comprehension:

The second critical challenge that this innovation takes on pertains to the imperative enhancement of the invention grasp on syntactic dependencies. These dependencies are the intricate threads weaving together the relationships between words within a sentence, governing their grammatical and structural roles. This understanding holds monumental importance across a diverse spectrum of NLP applications, ranging from machine translation and text summarization to sentiment analysis and question-answering systems.

However, the labyrinthine intricacies of human language and the myriad ways sentences can be structured create a formidable obstacle in capturing these dependencies with precision. The innovation rises to this challenge by embarking on the invention to enrich the invention comprehension and seamless integration of syntactic dependencies. This effort, 5 in turn, is anticipated to pave the way for language models tailored for clinical NLP that are more accurate and reliable.

In essence, this innovation's overarching aspiration is to reframe and elevate the capabilities of language models within the domain of clinical contexts. By adroitly addressing the instability of word embeddings and intensifying the grasp of syntactic dependencies, the innovation sets out to fortify the accuracy and efficacy of natural language processing applications within the clinical arena. This, in turn, has the potential to reshape and revolutionize crucial domains such as medical research, diagnosis, and healthcare practices, as language models gain an enhanced ability to interpret and analyse complex clinical narratives with greater precision.

### Applicability of the Present Invention:

Clinical Decision Support Systems (CDSS): The language model can be integrated into CDSS to aid healthcare professionals in making informed decisions. By comprehending and accurately representing clinical narratives, the system can offer context-aware suggestions and insights, helping doctors to formulate more precise diagnoses and treatment plans.
Electronic Health Records (EHR) Enhancement: The invention can enhance the functionality of EHR systems by improving their ability to interpret and organize clinical notes. It could assist in auto-populating relevant sections, extracting key information, and ensuring more accurate and comprehensive records.
Medical Coding Automation: Accurate medical coding is vital for billing and insurance purposes. The language model could facilitate automated coding by identifying relevant diagnoses, procedures, and other medical information within clinical notes, reducing manual effort and potential errors.
Clinical Research and Analysis: Researchers could utilize the invention to analyse large volumes of clinical data, extracting meaningful insights and patterns. This could aid in identifying trends, conducting retrospective studies, and contributing to medical research.
Healthcare Chatbots: Incorporating the language model into healthcare chatbots could enhance their understanding of patient queries and medical history. The system could provide more relevant responses and even triage patients based on their symptoms and medical context.
Medical Documentation Improvement: The invention could help healthcare providers improve the quality of their clinical documentation. By assisting in generating comprehensive and accurate notes, it could contribute to better communication among medical teams and potentially lead to improved patient care.
Telemedicine: In telemedicine scenarios, where direct patient interactions might be limited, the invention could aid in understanding and analysing patient-reported symptoms, making virtual consultations more effective.
Medical Education and Training: The invention could be employed in medical education to help trainees better understand complex clinical narratives. It could assist in teaching medical terminology, diagnosing skills, and the nuances of patient history analysis.
Clinical Trials: The language model could assist in screening and selecting suitable candidates for clinical trials by analysing patient profiles and medical history for eligibility criteria.
Healthcare Compliance and Auditing: By accurately extracting and representing clinical information, the invention could contribute to healthcare compliance and auditing processes, ensuring accurate billing and adherence to regulatory guidelines.

These abilities highlight the potential of the described invention to revolutionize various aspects of healthcare and medical practice by enabling more effective and efficient utilization of clinical language data. Although embodiments for the present subject matter have been described in language specific to structural features, it is to be understood that the present subject matter is not necessarily limited to the specific features described.

Rather, the specific features and methods are disclosed as embodiments for the present subject matter. Numerous modifications and adaptations of the system/component of the present invention will be apparent to those skilled in the art, and thus it is intended by the appended claims to cover all such modifications and adaptations which fall within the scope of the present subject matter.

We claim:

1. An advanced NLP-powered language modeling method for extracting information for the clinical domain, comprising:
collecting a clinical dataset in clean electronic format from a number of clinical notes collected from one or more sources;
developing a training corpus through the clinical dataset;
extracting profiling factors, from the clinical dataset, of user to avoid bias in the training corpus;
constructing n-grams, through the training datasets, by employing a unified medical language system (UMLS) as a guiding framework, to encode contextual information of the clinical dataset;
implementing pre-trained Bidirectional Encoder Representations from Transformers (BERT) for in-sentence prediction and masked language modelling; and
segmenting sentences based on pre-defined criteria, through tokenizers, into coherent units to identify irregularity in sentence structure.

2. The method as claimed in claim 1, wherein the profiling factors include but are not limited to geographic location of the patient, age, gender and patient type.

3. The method as claimed in claim 1, wherein the one or more sources include but are not limited to clinical notes.

4. The method as claimed in claim 1, wherein the n-grams may include but are not limited to bigram.

5. The method as claimed in claim 1, wherein the n-grams encapsulate the intricate relationship and terminologies inherent to clinical narratives.

6. The method as claimed in claim 1, wherein the criteria for the sentence segmentation include but are not limited to double line space.

7. A system for implementing the method as claimed in claim 1, the system comprising:
an input module for receiving inputs from one or more sources to generate the training corpus from the clinical dataset; and
one or more processors connected to the input module, and configured to:
extract relevant n-grams from the training corpus to encode information mentioned on the clinical notes;
implement the Bidirectional Encoder Representations from Transformers (BERT) for next-sentence prediction and masked language modelling (MLM); and
segment the sentences into coherent units for sentence demarcation.
